(19) United States
(12) Patent Application Publication
(10) Pub. No.: US 2002/0103526 A1
(43) Pub. Date: Aug. 1, 2002

(54) Protective coating for stent

(76) Inventor: Tom Steinke, San Diego, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 10/017,341

(22) Filed: Dec. 13, 2001

Related U.S. Application Data
(60) Provisional application No. 60/255,995, filed on Dec. 15, 2000.

(51) Int. Cl. A61F 2/06

(52) U.S. Cl. 623/1.11

(57) ABSTRACT

The following invention discloses a coating for a stent which protects the stent during handling and insertion of the stent into a body lumen, prevents movement of the stent on the catheter delivery system during insertion, and dissolves or degrades to allow stent deployment.

### RELATED APPLICATIONS

This application claims priority under 35 U.S.C. §119(e) to U.S. Provisional Application No. 60/255,995 filed on Dec. 15, 2000.

### BACKGROUND OF THE INVENTION

1. Field of the Invention

This invention relates to a class of expandable medical implants known as “stents” which are used to maintain support of a body lumen. More specifically, the invention discloses the use of a polymeric coating in conjunction with a stent delivery system.

2. Description of the Related Art

An important use of stents is found in situations where part of the vessel wall or stenotic plaque blocks or occludes blood flow in the vessel. Often, a balloon catheter is utilized in a percutaneous transluminal coronary angioplasty (PTCA) procedure to enlarge the occluded portion of the vessel. However, the dilation of the occlusion can cause fissuring of atherosclerotic plaque and damage to the endothelium and underlying smooth muscle cell layer, potentially leading to immediate problems from flap formation or perforations in the vessel wall, as well as long-term problems with restenosis of the dilated vessel.

To address these problems, implantation of stents can provide support for the body vessel, prevent re-closure of the vessel or provide patch repair for a perforated vessel. Further, the stent may overcome the tendency of diseased vessel walls to collapse, thereby maintaining a more normal flow of blood through that vessel.

A stent usually consists of a tubular structure that expands radially to be implanted into the tissue surrounding a body vessel. The stent is delivered to the site of implantation by means of a stent delivery system, which usually includes a catheter that supports the stent in a radially collapsed state for transport to the implantation site.

Stents can be deployed in a body lumen by means appropriate to their design. One such method would be to fit the collapsed stent over an inflatable element of a balloon catheter and expand the balloon to force the stent into contact with the body lumen. As the balloon is inflated, the problem material in the vessel is compressed in a direction generally perpendicular to the wall of the vessel which, consequently, dilates the vessel to facilitate blood flow therethrough. Radial expansion of the coronary artery occurs in several different dimensions and is related to the nature of the plaque. Soft, fatty plaque deposits are flattened by the balloon and hardened deposits are cracked and split to enlarge the lumen. It is desirable to have the stent radially expand in a uniform manner.

Alternatively, the stent may be mounted onto a catheter that holds the stent as it is delivered through the body lumen and then releases the stent and allows it to self-expand into contact with the body lumen. This deployment is effected after the stent has been introduced percutaneously, transported transluminally and positioned at a desired location by means of the catheter.

Several difficulties have been encountered in the handling and insertion of stents. First, due to the delicate nature and small size of the stent, there is a potential for damage of the stent during handling by the physician prior to insertion. Another common problem with stent deployment is slippage and early unintentional release of the stent, as discussed in U.S. Pat. No. 5,830,217 issued to Ryan. The stent may pop off the balloon during inflation or may slip backward off the balloon during steering to the intended site of release. In addition, passage of the stent through the hemostasis valve often causes the stent to be damaged or dislodged from the stent delivery device. Displaced stents often require removal of the stent from the body, a process which can result in severe damage to the body vessel and can require invasive surgery to remove the stent.

As a result, there is a need for a stent delivery system which can, among other things, protect the stent during handling of the stent prior to insertion, can prevent movement of the stent on the delivery catheter during insertion of the stent into a body vessel, and can be removed to allow the stent to be implanted at the deployment site.

### SUMMARY OF THE INVENTION

In one embodiment of the present invention, a protective coating is disclosed for a stent, which protects the stent from damage during handling of the stent prior to insertion and during insertion of the stent into a body lumen. The coating prevents movement of the stent on a delivery catheter during handling and insertion. The coating also provides a lubricious surface that aids in endoluminal navigation. The coating consists of a dissolvable or degradable polymer.

The coating can also be loaded with therapeutic compounds that can be delivered when the stent assembly is delivered to its desired location.

### BRIEF DESCRIPTION OF THE DRAWINGS

FIG. 1 is a perspective view of a representative stent.

FIG. 2 is a perspective view of a typical balloon-mounted stent assembly system used for stent delivery.

FIG. 3 is a plan view of a variation of the stent showing the linkage of adjacent modules, each comprising alternating one-rib and two-rib radial elements, wherein the one-rib radial elements further comprise a frame element adapted to facilitate linkage of adjacent modules in the circumferential axis.

FIG. 4 is a perspective view of a stent with a polymeric coating.

### DETAILED DESCRIPTION OF THE PREFERRED EMBODIMENT

The subject matter disclosed in this application is related to that disclosed in copending U.S. patent application Ser. No. 09/283,800 filed on Apr. 1, 1999, now U.S. Pat. No. 6,224,626, which is a continuation-in-part of U.S. patent application Ser. No. 09/024,571 filed on Feb. 17, 1998, now U.S. Pat. No. 6,033,436; which are incorporated herein in their entirety by reference thereto. This application is also related to a co-pending U.S. patent application Ser. No. 09/739,552, entitled “EXPANDABLE STENT WITH SLIDING AND LOCKING RADIAL ELEMENTS” to Steinke et al., filed on Dec. 14, 2000; which is incorporated herein in its entirety by reference thereto.

Several approaches have been proposed for delivering and deploying a stent with a protective sheath or coating. For example, U.S. Pat. No. 5,445,646 to Euteneuer, which is incorporated in its entirety herein by reference thereto, discloses a delivery system for implantation of a self-expanding stent in a vessel where the stent is held in a reduced delivery configuration for insertion and transport through a body lumen to a predetermined site for deployment. Several embodiments in which sleeves are used to hold the stent in its reduced delivery configuration are disclosed. The stent may be held in the delivery configuration by means of a tubular sleeve made of water-soluble material, a plurality of bands made of water-soluble material, swelling band(s), or other degradable material.

U.S. Pat. No. 5,989,280 to Euteneuer, which is incorporated in its entirety herein by reference thereto, discloses a stent delivery device where the stent is held in a reduced delivery configuration for insertion and transport through a body lumen to a predetermined site for deployment of a stent, self-expanding stent, stent graft or the like. The use of sleeves which can either hold a self-expanding stent in the delivery configuration or form a watertight chamber for an enclosed holding means are disclosed. Balloon expandable stents may also be delivered within a sleeve. Disadvantageously, the use of sleeves increases the overall diameter of the stent device.

U.S. Pat. No. 5,830,217 to Ryan, which is incorporated in its entirety herein by reference thereto, discloses a soluble capsule, fairing, or adherent material for a stent catheter or other device to be inserted into the human body. The distal end of the catheter, including the stent or at least some portion of the stent, is coated with a bioabsorbable and quickly dissolvable material. This material encapsulates the catheter deployed device, providing a smooth surface for the device during passage through the vasculature. The preferred bioabsorbable material is a polysaccharide; however, other polymers including proteins, lipids, and synthetic compositions such as biocompatible polymers are also disclosed. The material can be used with self-deploying stents. It can also be mixed with other compounds which provide pain killers, anti-coagulants, and anti-thrombus agents.

U.S. Pat. No. 5,637,113 to Tartaglia, which is incorporated in its entirety herein by reference thereto, discloses an expandable stent structural member and a planar sheet of polymeric material disposed on the outside of the expandable stent structural member. The polymeric material is preferably bioabsorbable, and is preferably loaded or coated or laminated with a therapeutic agent or drug to reduce or prevent restenosis and thrombosis in the vessel being treated. The polymer film can be attached to the existing stent structural member in an unexpanded state by adhesive, by heat sealing, or mechanically. The polymer material can be attached to the stent structural member at one or more points and wrapped in a coil around the stent in an unexpanded state. The polymer material can also be attached to an existing stent structural member by an interference fit by tightly wrapping the material around the stent structural member in an unexpanded state and attaching the polymer film to itself to form a sleeve around the stent structural member. In one embodiment, the stent structural member and polymeric film wrapping are provided with an additional coating of lubricious material, which facilitates insertion of the stent through the vasculature by providing a low friction surface over the stent. Disadvantageously, Tartaglia does not teach the use of a polymeric material which can be used to prevent movement of the stent on the catheter during handling and insertion.

U.S. Pat. No. 5,234,457 to Andersen, which is incorporated in its entirety herein by reference thereto, describes a stent assembly system comprising a compact mesh in a cylindrical form. A cured dissolvable material impregnates the mesh and contains the mesh in its compact form during placement. The cured material dissolves when the stent is in position in the body thereby to free the mesh and enable its expansion into a final form contacting the tissue surrounding the vessel. Disadvantageously, Andersen does not teach the use of a dissolvable material which can secure the stent on the catheter during handling and insertion into the body.

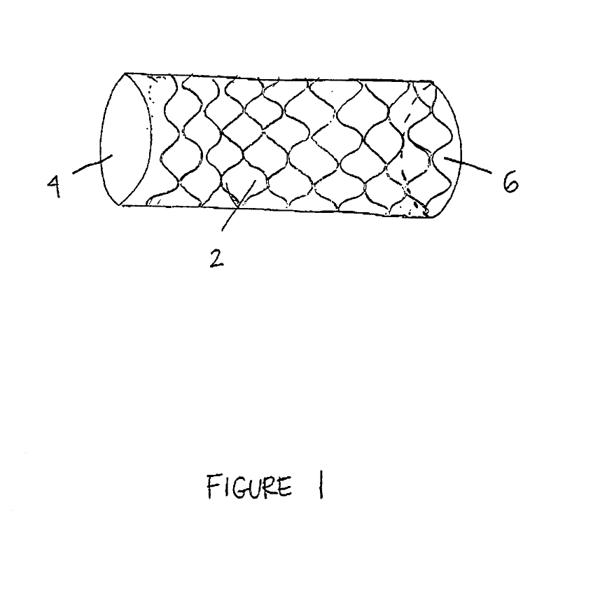

As depicted in FIG. 1, a typical stent 2 used for implantation in a body lumen has a tubular structure with a distal end 4 and a proximal end 6. The illustrated stent is depicted with a sinusoidal support structure, but the invention can also be used with other forms of stent support structures, including the expandable stents disclosed in U.S. Pat. Nos. 6,033,436 and 6,224,626 issued to Steinke.

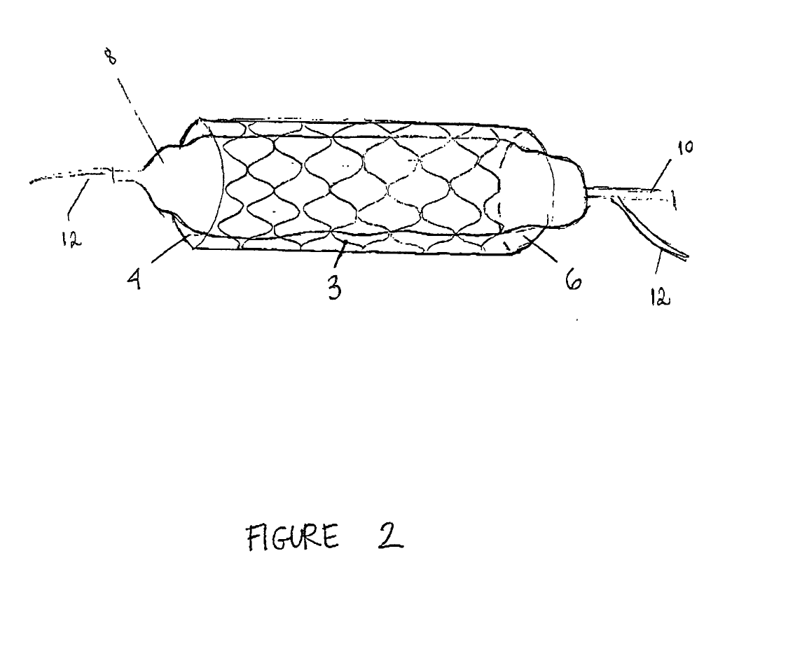

The stent 2 can be radially expanded from an initial, compressed state to an expanded, deployed state. In a typical balloon-mounted stent assembly depicted in FIG. 2, the compressed stent 3 is mounted onto a balloon 8, which is in turn mounted on a catheter 10. A guidewire 12 typically extends from the distal end 4 of the catheter 10 running proximally through the catheter 10 to the exit point just proximal 6 of the balloon 8. The stent 3 expands into a deployed state at the site of implantation.

Alternate stent designs may also be utilized such as a radially expanding stent comprising a tubular member with a clear through-lumen. The tubular member has proximal and distal ends and a longitudinal length defined therebetween, and a circumference, and a diameter which is adjustable between at least a first collapsed diameter and at least a second expanded diameter. In a preferred mode, the longitudinal length remains substantially unchanged when the tubular member is adjusted between the first collapsed diameter and the second collapsed diameter. The tubular member includes at least one module comprising a series of sliding and locking radial elements, wherein each radial element defines a portion of the circumference of the tubular member and wherein no radial element overlaps with itself in either the first collapsed diameter or the second expanded diameter.

In one aspect, each radial element may comprise at least one elongated rib disposed between first and second end portions. Preferably, the radial elements that comprise a module alternate between radial elements having an odd number of elongated ribs and radial elements having an even number of elongated ribs. In one preferred mode, the radial elements alternate between radial elements having one elongated rib and radial elements having two elongated ribs.

In one preferred embodiment, the stent also includes at least one articulating mechanism comprising a tab and at least one stop. The articulating mechanism permits one-way sliding of the radial elements from the first collapsed diameter to the second expanded diameter, but inhibits radial recoil from the second expanded diameter.

In variations to the stent, the tubular member may comprise at least two modules which are coupled to one another by at least one linkage element. In one variation, the tubular member may further comprise a frame element that surrounds at least one radial element in each module. In stents in which the tubular member comprises at least two modules, such frame elements from adjacent modules may be coupled. The coupling may include a linkage element extending between the frame elements. In addition or in the alternative, the frame elements from adjacent modules may be coupled by interlinking of the frame elements. In another aspect, the intermodular coupling may be degradable allowing for the independent modules to adapt to the vessel curvature.

In another variation to the stent of the present invention, any amount of overlap among the radial elements within a module remains constant as the tubular member is adjusted from the first collapsed diameter to the second expanded diameter. This amount of overlap is preferably less than about 15%.

The radial recoil of the tubular member in accordance with one preferred embodiment is less than about 5%. The stiffness of the stent is preferably less than about 0.01 Newtons force/millimeter deflection. The tubular member preferably provides a surface area coverage of greater than about 20%.

In accordance with another variation of the present stent, the tubular member is at least partially radiopaque. The radial elements may be made substantially from a material which is work hardened to between about 80% and 95%. In one preferred variation, the radial elements in the expandable intraluminal stent are made from a material selected from the group consisting of a polymer, a metal, a ceramic, and combinations thereof. In one mode, the material may be degradable.

In another mode of the invention, the material may also include a bioactive agent. The material is preferable adapted to deliver an amount of the bioactive agent which is sufficient to inhibit restenosis at the site of stent deployment. In one variation, the radial elements are adapted to release the bioactive agent during stent deployment when the tubular member is adjusted from the first collapsed diameter to the second expanded diameter. The bioactive agents are preferably selected from the group consisting of antiplatelet agents, antithrombin agents, antiproliferative agents, and antiinflammatory agents.

In another variation, the tubular member further comprises a sheath, such as for example in a vessel graft.

In one aspect, the expandable intraluminal stent comprises at least two modules, wherein the expanded diameters of the first and second modules are different. The articulating mechanisms of the present invention which allow the stent to expand but inhibit stent recoil, may comprise a slot and a tab on one radial element and at least one stop on an adjacent radial element which is slideably engaged in the slot, wherein the tab is adapted to engage the at least one stop. The articulating mechanisms may also include an expansion resistor on the slideably engaged radial element, wherein the expansion resistor resists passing through the slot during expansion until further force is applied, such that the radial elements in the module expand in a substantially uniform manner. In another variation, the articulating mechanism may include a release, such that actuation of the release permits sliding of the radial elements from the second expanded diameter back to the first collapsed diameter for possible removal of the stent.

In another variation, the stent may comprise a floating coupling element having an articulating mechanism.

In another variation, the expandable intraluminal stent comprises a tubular member with a clear through-lumen and a diameter which is adjustable between at least a first collapsed diameter and at least a second expanded diameter. The tubular member comprises a series of sliding and locking radial elements made from a degradable material, wherein each radial element in the series defines a portion of the circumference of the tubular member and wherein no radial element overlaps itself. This stent also has at least one articulating mechanism that permits one-way sliding of the radial elements from the first collapsed diameter to the second expanded diameter, but inhibits radial recoil from the second expanded diameter. The degradable material may be selected from the group consisting of polyarylates (L-tyrosine-derived), free acid polyarylates, polycarbonates (L-tyrosine-derived), polyester-amides), polypropylene fumarate-co-ethylene glycol) copolymer, polyanhydride esters, polyanhydrides, polyorthoesters, and silk-elastin polymers, calcium phosphate, magnesium alloys or blends thereof.

In a variation to the degradable stent, the degradable polymer may further comprise at least one bioactive agent, which is released as the material degrades. The at least one bioactive agent may be selected from the group consisting of antiplatelet agents, antithrombin agents, antiproliferative agents and antiinflammatory agents.

In another variation, the stent material may be fiber-reinforced. The reinforcing material may be a degradable material such as calcium phosphate (e.g., BIOGLASS). Alternatively, the fibers may be fiberglass, graphite, or other non-degradable material. In another mode, the stmt of the present invention comprises a tubular member having a wall and a clear through-lumen. The tubular member comprising a series of sliding and locking radial elements which do not overlap with themselves. The radial elements further comprise a ratcheting mechanism that permits one-way sliding of the radial elements from a first collapsed diameter to a second expanded diameter. The tubular member in this embodiment has a stiffness of less than about 0.01 Newtons force/millimeter deflection, and the wall of the tubular member has a thickness of less than about 0.005 inches.

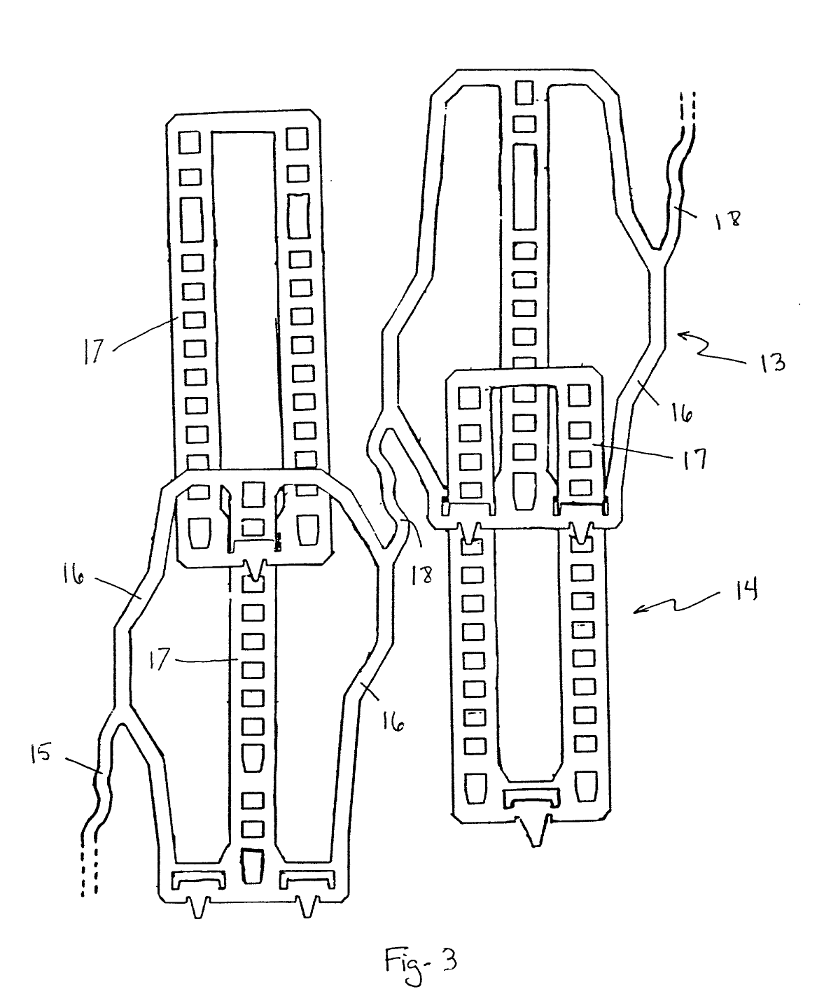

The shape of the frame elements can be varied to cause circumferential off-setting of the different radial elements having odd and even-numbers of ribs. For example, with reference to FIG. 3, the lateral coupling of one pair of radial elements (a one-rib 13 and a two-rib 14 radial element) from one module are connected by the linkage element 18 to another pair of radial elements from an adjacent module. The frame elements 16 are shown in this embodiment surrounding only the one-rib radial elements 13. The frame elements 16 are configured so as to promote nesting (and not overlap) of ribs 17 and frame elements 16, minimize the lateral space between the modules, and facilitate linkage by a circumferentially, rather than longitudinally, oriented linkage element 16, thereby maximizing the circumferential scaffolding and radial support.

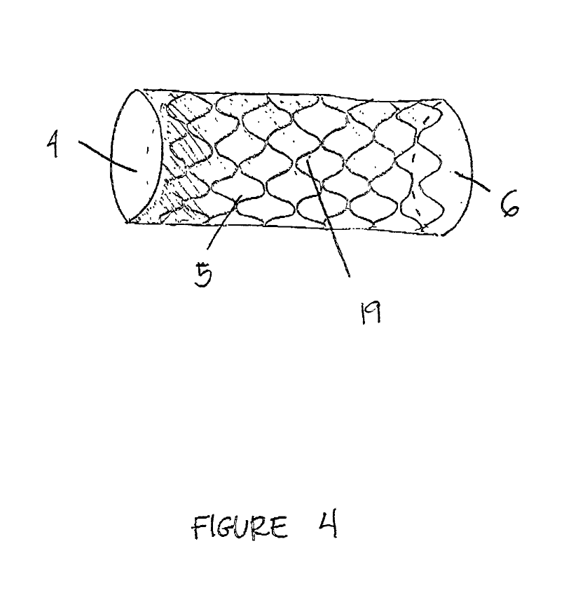

In one embodiment of the current invention, which is illustrated in FIG. 4, a stent 5, such as that described with reference to FIG. 3, is covered with a protective coating 19. The material used to form the protective coating 19 can include, but is not limited to, polymeric materials such as polyvinyl pyrrolidone, polyethylene glycol, polyethylene oxide, polyethylene acetate, polyvinyl alcohol, polyvinyl acetate, polyacrylic acid, polypropylene oxide, polymethacrylic acid, polyacrylamide, hydrophilic soft segment urethane, gum Arabic, gum tragacanth, latexes, polyanhydrides, ethylene vinyl acetate, polysiloxanes, modified styrene-ethylene/butylene-styrene block polymers, aliphatic polyesters, resorbable polyesters or any combination thereof. Examples are poly(propylene fumarate-coethylene glycol) copolymer (aka fumarate anhydrides), polyanhydride esters, polyanhydrides, polyaspartimic acid, polycaprolactone, polyglycolic acids or copolymers thereof, polyorthesters, polyphosphazenes, tyrosine polyarylates, tyrosine polycarbonates (e.g., poly(3-(4-hydroxyphenyl)propionyl tyrosine [ethyl ester] carbonate) a.k.a. poly(desaminotyrosyl tyrosine [ethyl ester] carbonate)) or copolymers such as poly(75%DTE-co-25%DT carbonate)s, polyamids. More slowly degrading polymers are poly lactic acids or co-polymers thereof (e.g., lactic acid/ethylene glycol copolymers), poly(ester amide)s, polydioxanone or poly-p-diaxanone) or PHV/PHB (i.e., polyhydroxybutyrate/polyhydroxyvalerate copolymers).

Other types of materials that are based on natural materials may be used and include polysaccharides such as sucrose, mannitol, sorbitol, xylitol, fructose, dextrose, glucose, glucosamine, lactose, and combinations thereof. Anionic hydrated polysaccharides may be used such as gellan, curdlan, XM-6, xanthan, and combinations thereof. Seaweed polysaccharides may be used such as agar, algin, carrageenan, furcelleran and combinations thereof. Cellulose derivatives such as alkyl cellulose, hydroxymethyl cellulose and combinations thereof may be used. Alginates, calcium phosphate glass alone or with other resorbable polymers, chitosan (e.g., NOOC or NOOC-G), collagen, fibrin or fibrinogen, hyaluronic acid, hydroxy acids (i.e. lactide, glycolide, hydroxybutyrate), lactone-based polymers, or even silk-elastin polymers.

Other useful materials include pectins, gels, gelatin, soluble starches, mucoid substances, dextrans, dextranes, dextrins and combinations thereof. Lipid-based coatings may also be used.

The protective coating is preferably dissolvable or degradable (by hydrolysis which is water altering the chemical linkages or by biodegradation e.g., through the action of biological enzymes altering the chemical linkages) within the body. As a result, it serves to protect the stent during handling and insertion in the body, but it dissolves in blood or degrades to allow the stent to be deployed at the implantation site.

In one method of preparing the coating, the material used for the coating is first dissolved in a solvent, such as 100% ethanol or other alcohols for instance methanol or isopropanol, as well as other solvents including but not limited to water, diethylene glycol, methylene chloride, chloroform, polyethylene glycol, glycerol, dimethyl formamide, tetrahydro-furan, hexafluoro-isopropanol or other Class 2 or 3 solvents regarded as acceptable for pharmaceutical applications and that are compatible with the coating substance. to obtain a solution which is 20% concentration by weight. The solution is then applied to the stent by using techniques known to those skilled in the art, such as the process disclosed in U.S. Pat. No. 5,234,457, which is hereby incorporated by reference, in which the stent is rotated on a mandrel as the solution is poured over the stent. The stent is then air cured to form the protective coating.

The coating may be applied manually with the aid of a calibrated dispenser or through controlled spray-coating or dipping process. Once applied the substance the coating may be cured by allowing the solvent to evaporate and leave the dried coating on the stent. Alternatively the coating might by light cured, depending upon its chemical nature. The thickness of the coating ranges from about 0.001″-0.0015″ (about 25.4-38.1 microns thick).

When the protective coating is formed, it protects the stent from damage during handling of the stent prior to insertion. The coating also prevents stent movement on the delivery catheter during insertion. The coating forms a thin layer in and around the stent and the balloon, temporarily bonding the two together. Once wetted, either during stent preparation or after direct exposure to the blood, the very thin coating absorbs water creating a more pliable and/or lubricous surface coating.

The coating can also be used to deliver other compounds along with the stent. These compounds include, but are not limited to, anticoagulants (e.g., heparin), antithrombotics, antiplatelets (e.g., abciximab), cytostatic and antiproliferative agents (e.g., rapamycin, taxol, C6-ceramide), or other moieties that bind to the family of intracellular receptors named FKBPs (e.g., RAD), antiinflammatory (e.g., dexamethasone, methyl prednisolone), antimitogens, antimitotoxins, antioxidants (e.g., probucol), lipid regulating (e.g., pravastatin, pitavastatin), antisense oligonucleotides (e.g., c-myc), gene therapy vehicles, nitric oxide, growth factors and inhibitors, hirudin, hirugen, hirulog, anti-migratory drug (e.g., batimastat), ace inhibitors (e.g., cilazapril, tranilast), somatostatin analogues (e.g., angiopeptin), other statins (e.g., simvastatin, lovastatin, fluvastatin, lovastatin), vasodilators (e.g., trapidil), tacrolimus vincristine, argatroban, forskolin, vapiprost, prostacyclin and prostacyclin analogues, dextran, dipyridamole, glycoprotein IIb/IIIa, platelet membrane receptor antibody, recombinant hirudin, thrombin inhibitor, angiopeptin, angiotensin converting enzyme inhibitors (such as Captopril (Squibb), Cilazapril (Hoffman-La Roche) or Lisinopril (Merck)), calcium channel blockers, colchicine, fibroblast growth factor antagonists, fish oil, omega 3-fatty acid, histamine antagonists, HMG-CoA reductase inhibitor, methotrexate, monoclonal antibodies, nitroprusside, phosphodiesterase inhibitors, prostaglandin inhibitor, seramin, serotonin blockers, steroids, thioprotease inhibitors, triazolopyrimidine and other PDGF antagonists, alpha-interferon, genetically engineered epithelial cells, tyrosine kinase inhibitor, D-Pro-Phe-Arg chloromethyl ketone (PPACK), D-phenylalanyl-L-prolyl-L-arginyl chloromethyl ketone (FPRCH2Cl), warfarin, and combinations thereof. The compounds can be added to the coating using techniques known to those skilled in the art, such as the techniques described in U.S. Pat. No. 5,234,457 issued to Andersen, U.S. Pat. No. 5,830,217 issued to Ryan, and U.S. Pat. No. 5.989,280, issued to Euteneuer, all of which are hereby incorporated by reference. The drug delivery release may occur by degradation or biodegradation of the coating, diffusion of the drug out of the coating, or through time-controlled release.

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A method for applying a protective coating for a stent delivery system, comprising:
selecting a dissolvable or degradable polymer;
dissolving said polymer into a solvent to make a solution;
applying said solution to the surface of the stent delivery system comprising a stent and an expandable member;
allowing said solution to air cure to form a coating around the stent and the expandable member, wherein said stent and said expandable member are bonded together.

2. The method of claim 1 wherein the polymer is dissolvable in blood.

3. The method of claim 1, wherein the solution is manually applied to the stent and the expandable member.

4. The method of claim 1, wherein the solution is applied by controlled spray-coating.

5. The method of claim 1, wherein the solution is applied by a dipping process.

6. The method of claim 1, wherein the thickness of the coating is from about 0.001 inches to 0.0015 inches.

7. The product formed by the process of claim 1.

8. The method of claim 1, wherein said polymer is selected from a group consisting of polyvinyl pyrrolidone, polyethylene glycol, polyethylene oxide, polyethylene acetate, polyvinyl alcohol, polyacrylic acid, polymethacrylic acid, polyacrylamide, hydrophilic soft segment urethane, gum Arabic, gum tragacanth, C6-ceramide, or any combination thereof.

9. The method of claim 1, where said coating further comprises a compound selected from the group consisting of antithrombotics, anticoagulants, antimitogens, antimitotoxins, antisense oligonucleotides, gene therapy vehicles, nitric oxide, growth factors and inhibitors, hirudin, hirugen, hirulog, D-Pro-Phe-Arg chloromethyl ketone (PPACK), D-phenylalanyl-L-prolyl-L-arginyl chloromethyl ketone (FPRCH2Cl), heparin, C6-ceramide and warfarin.

10. A stent delivery system, comprising:
an elongated catheter having a proximal and distal end portion and an expandable member disposed along the distal end portion of the elongated catheter, said expandable member being coupled to an expansion actuator;
a stent which is adjustable between a first collapsed diameter and at least a second expanded diameter, comprising a tubular member having a length and a diameter, and comprising a series of sliding and locking radial elements, and at least one articulating mechanism which permits one-way sliding of the radial elements from the first collapsed diameter to the second expanded diameter but inhibits radial recoil from the second expanded diameter, wherein said stent is disposed in its collapsed state over the expandable member on the elongated catheter; and
a degradable polymeric coating selected from a group consisting of polyvinyl pyrrolidone, polyethylene glycol, polyethylene oxide, polyethylene acetate, polyvinyl alcohol, polyacrylic acid, polymethacrylic acid, polyacrylamide, hydrophilic soft segment urethane, gum Arabic, gum tragacanth, or any combination thereof, wherein said polymeric coating holds said stent on said expandable member.

11. The stent delivery system of claim 10, where said coating further comprises a compound selected from the group consisting of antithrombotics, anticoagulants, antimitogens, antimitotoxins, antisense oligonucleotides, gene therapy vehicles, nitric oxide, growth factors and inhibitors, hirudin, hirugen, hirulog, D-Pro-Phe-Arg chloromethyl ketone (PPACK), D-phenylalanyl-L-prolyl-L-arginyl chloromethyl ketone (FPRCH2Cl), heparin, C6-ceramide and warfarin.

12. The stent delivery system of claim 10, wherein each radial element comprises at least one elongated rib disposed between first and second end portions.

13. The stent delivery system of claim 12, wherein the radial elements alternate between radial elements having an odd number of elongated ribs and radial elements having an even number of elongated ribs.

14. The stent delivery system of claim 13, wherein the radial elements alternate between radial elements having one elongated rib and radial elements having two elongated ribs.
